(19) United States
(12) Patent Application Publication
(10) Pub. No.: US 2025/0367073 A1
(43) Pub. Date: Dec. 4, 2025

(54) Automated Perineal Care System

(71) Applicant: Carnegie Mellon University, Pittsburgh, PA (US)

(72) Inventors: Herman HERMAN, Gibsonia, PA (US); Gabriel Jacob GOLDMAN, Pittsburgh, PA (US); Halit Bener SUAY, Pittsburgh, PA (US); Matthew Feld GLISSON, Pittsburgh, PA (US); Jose GONZALEZ-MORA, Pittsburgh, PA (US)

(21) Appl. No.: 18/707,951

(22) PCT Filed: Nov. 9, 2022
(86) PCT No.: PCT/US2022/079520

§ 371 Date: May 7, 2024

Related U.S. Application Data
(60) Provisional application No. 63/277,742, filed on Nov. 10, 2021.

(51) Int. Cl. A61H 35/00; A61F 13/84; A61G 9/02; A61H 33/00; A61H 33/02

(52) U.S. Cl. CPC A61H 35/00; A61F 13/84; A61G 9/02; A61H 33/005; A61H 33/026; A61H 33/6021; A61H 2201/0103; A61H 2201/0142; A61H 2201/0161; A61H 2201/1623; A61H 2201/164; A61H 2203/0437; A61H 2205/08

(57) ABSTRACT

Systems, apparatuses and methods for automated perineal care are provided. A system embodiment includes a user support element, a sensor configured to measure a cleanliness state of a user, a cleaning element configured to clean a user perineal area, and a controller configured to command operation of the cleaning element based on the sensor. An apparatus embodiment includes a containment structure, a sensor, a cleaning element assembly including a drive, actuator and a nozzle, and a controller configured to control the cleaning element assembly based on the sensor. A method embodiment includes positioning a user into a perineal cleaning position, scanning the user with a sensor, determining a cleanliness status, and activating a cleaning element based on the cleanliness status.

### CROSS-REFERENCE TO RELATED APPLICATIONS

This application is a U.S. National Stage Application under 35 U.S.C. § 371 of International Patent Application No. PCT/US2022/079520, filed Nov. 9, 2022, which claims the benefit of U.S. Provisional Patent Application No. 63/277,742, filed Nov. 10, 2021, both of which are incorporated herein by reference in their entireties.

### TECHNICAL FIELD

The relevant technical field includes perineal care giving and automated machine assisted hygiene.

### BACKGROUND

The perineal area of a human body is prone to odors and infection and must be cleaned periodically. Such cleaning is particularly important for individuals suffering from incontinence or lacking the ability to independently maintain personal perineal hygiene. Care giver assisted perineal cleaning is a resource intensive activity and generates significant mental stress, both on care givers and patients.

### SUMMARY

A system of automated perineal care comprises a user support element, a sensor positioned on the support element and configured to measure a cleanliness state of a user, a cleaning element configured to clean a perineal area of a user, and a controller configured to command the cleaning element based on a signal from the sensor.

An apparatus for automated perineal care comprises a containment structure forming an interior compartment having a drain, a sensor mounted to the containment structure, a cleaning element assembly comprising a drive mounted to the containment structure and configured to provide travel access to the interior compartment, an actuator connected to the drive and separated from the interior compartment by the containment structure, a nozzle carriage connected with the drive and including a nozzle configured to emit a liquid or gas, and a controller configured to the control the cleaning element assembly based on a signal from the sensor.

An apparatus for perineal care comprises a containment structure forming an interior compartment, a first and second rotating support mounted to the containment structure and positioned adjacent to the interior compartment, and a first diaper manipulator mounted to the first support and a second diaper manipulator mounted to the second support, the diaper manipulators configured to remove a diaper article by rotational contact.

An apparatus for perineal care positioning comprises a linear motion track, a back support structure connected to the track by a first sliding attachment, a back support link connected to the back support structure by a first joint and connected to the track by a fixed attachment, and a foldable leg support structure formed by a first and second leg support link attached to one another by a second joint, the first leg support link connected to the track by a second sliding attachment and the second leg support link connected to the track by a third sliding attachment, wherein an orientation of the back support structure and the foldable leg support structure is configured to be adjusted based on an adjustment to a linear position of the first, second, and third slidable attachments along the track.

A method of automated perineal care comprises the steps of positioning a user into a perineal cleaning position, scanning the user with one or more sensors, determining a user cleanliness status based on a sensor measurement, and activating a cleaning element based on the user cleanliness status.

A method of automated perineal care comprises the steps of determining a user spatial location, determining a cleaning instrument's spatial location relative to the user, processing an operator applied force on the cleaning instrument, and moving the cleaning instrument by actuator assistance in response to the operator applied force and within a safety space as defined by the user's spatial location.

### BRIEF DESCRIPTION OF THE DRAWINGS

FIG. 1 is a diagram depicting a system embodiment for automated perineal cleaning.

FIG. 2 is a diagram depicting an example embodiment of perineal cleaning algorithmic inputs and outputs.

FIG. 3A is a diagram depicting a second example embodiment of perineal cleaning algorithmic inputs and outputs.

FIG. 3B is a diagram depicting a third example embodiment of perineal cleaning algorithmic inputs and outputs.

FIG. 4A is an internal view of an example embodiment of an automated perineal cleaning apparatus.

FIG. 4B is an angled-profile view of an example embodiment of an automated perineal cleaning apparatus.

FIG. 5 depicts examples of a user in various perineal cleaning positions.

FIG. 6A depicts an example embodiment of an automated perineal cleaning apparatus including a cleaning nozzle, actuator, drive, and rotation shaft.

FIG. 6B depicts an example embodiment of perineal cleaning nozzles, drives, and rotation shafts.

FIG. 7A depicts an example embodiment of an automated perineal cleaning apparatus including a plurality of nozzles.

FIG. 7B depicts an example embodiment of the apparatus of FIG. 7A.

FIG. 8 depicts an internal view of an example automated perineal cleaning apparatus embodiment including a diaper manipulator, diaper pusher and diaper receptacle.

FIG. 9 depicts an example automated perineal cleaning apparatus embodiment including a diaper manipulator in various stages of operation.

FIG. 10 depicts an example embodiment of the system of FIG. 1.

FIG. 11 depicts example embodiments of the system of FIG. 10.

FIG. 12A depicts an example embodiment of an automated perineal cleaning apparatus including an electro-mechanical arm configured to work cooperatively

FIG. 12B depicts an example embodiment of the automated perineal cleaning apparatus of FIG. 12A.

FIG. 13A and 13B depict an example embodiment of an electro-mechanical arm.

FIG. 14 depicts an example embodiment of the electro-mechanical arm of FIGS. 13A and 13B.

FIG. 15A and 15B depict an example embodiment of a cleaning tool including a suction pad.

FIG. 16 depicts an example embodiment of a cleaning tool including a rotary wiper.

FIGS. 17A and 17B depict example embodiments of a user support element.

FIGS. 18A and 18B depict example embodiments of cleaning sensor arrangements.

FIG. 19 depicts an example schematic embodiment of a controller.

FIG. 20 is a flow diagram depicting a method of automated perineal cleaning.

FIG. 21 is a flow diagram depicting a second method of automated perineal cleaning.

FIG. 22 is a flow diagram depicting a third method of automated perineal cleaning.

FIG. 23 is a flow diagram depicting a fourth method of automated perineal cleaning.

### DETAILED DESCRIPTION

The following detailed description is provided to assist the reader in gaining a comprehensive understanding of the systems, apparatuses, and methods described herein. Accordingly, various changes, modifications, and equivalents of the systems, apparatuses, and methods described herein will be suggested and thus apparent to those having ordinary skill in the art. Also, descriptions of well-known functions and constructions may be omitted for increased clarity and conciseness to the reader.

In addition, it is to be understood that the phraseology and terminology employed herein are for the purpose of description and should not be regarded as limiting. For example, the use of a singular term, such as, “a” is not intended as limiting of the number of items. Also, the use of relational terms, such as but not limited to, “top,” “bottom,” “left,” “right,” “upper,” “lower,” “down,” “up,” “side,” are used in the description for clarity and are not intended to limit the scope of the invention or the appended claims. Further, it should be understood that any one of the features can be used separately or in combination with other features. Other systems, methods, features, and advantages of the invention will be or become apparent to one having ordinary skill in the art upon examination of the detailed description. It is intended that such additional systems, methods, features, and advantages be included within this description, be within the scope of the present invention, and be protected by the accompanying claims.

The region of the body superficial to the muscles of the pelvic floor and medial to the thighs is known as the perineal area. The perineal area is a source of various waste materials including urine and fecal matter and must be periodically cleaned to maintain hygiene and health. The perineal area may be cleaned by various strategies, in some instances including self-cleaning, care giver assisted cleaning, and semi or fully automated machine assisted cleaning.

Perineal cleaning includes several performance modes that, depending on the specific perineal cleaning function to be performed, may be utilized individually or in combination to complete a required perineal cleaning task. Broadly, perineal cleaning includes such modes as supporting an individual to be cleaned, identifying the presence or lack of waste material on the individual, and cleaning or removing said waste material from the individual.

An individual may be supported in various orientations so as to provide cleaning access to the perineal area. One such example orientation includes a lithotomy position, in which the upper body is supine while the knees are bent, and the legs are elevated. Other example orientations include standing, seating, squatting, laying, or otherwise bending over. An individual may be supported during perineal cleaning by various structures including a seat, a sling or hoist, a table, a bed, a support bar, an inflatable structure, or any other structure capable of supporting an individual in a specific position or orientation.

In embodiments, the presence or absence of waste material may be sensed using various approaches. Further, the location of said waste material may be identified in some instances so as to direct a cleaning or waste removal operation. Such waste material sensing may include not only information of where waste material exists in 3D space, but also where waste material exists relative to a body. Accordingly, it may be beneficial to also sense the location or dimension of a body to be cleaned. Examples of such waste material or body sensing methods include visual, electro-optical, laser, infra-red, thermal, acoustic, or any other method or technology capable of detecting or measuring waste material or an individual's body.

The perineal area may be cleaned by various approaches. Waste material can be removed using physical means such as liquid streams, gas streams, brushes, scrappers, wipes, or combinations thereof. Additionally, the perineal area may be cleaned by microbial disinfection, such as by introducing a disinfectant chemical, ultraviolet light source, or any other process for disinfecting or destroying an organic waste material or microorganism. In one example, the perineal area is cleaned using a stream of water or room air. In another example, the perineal area is cleaned using a cleaning chemical, solution or inert gas.

Numerous advantages and benefits may be realized by combining such described modes into systems, apparatuses, and methods capable of autonomous, semi-autonomous, or operator-assisted perineal area cleaning. Such advantages and benefits may include reduced care giver labor and mental stress, increased occupational safety thanks in part to better separation of care givers from biohazardous waste materials, reduced patient mental stress and embarrassment, and improved quality of perineal cleaning.

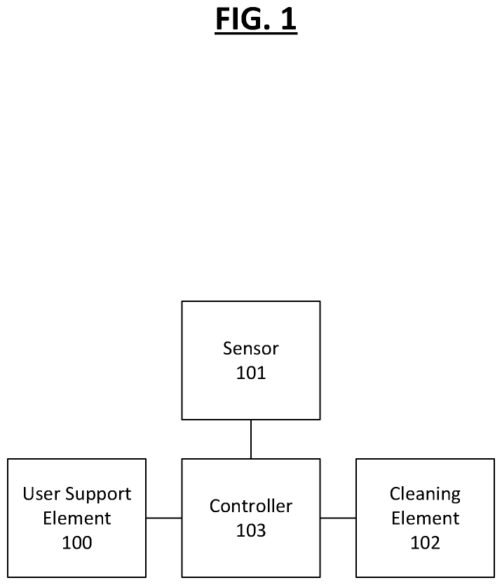

FIG. 1 depicts an example system embodiment for automated perineal cleaning. The system comprises an element 100 configured to support a user, a sensor 101 configured to scan and detect or measure a cleanliness state of a user, a cleaning element 102 configured to clean a perineal area of a user, and a controller 103 configured to operate or control operation of the cleaning element 102 based on a signal from sensors 101. In some embodiments of the system, the user support element 100 is a perineal positioning cleaning structure configured to provide cleaning access to a user's perineal area. The perineal positioning cleaning structure may comprise a support frame, surface, or inflatable structure and be configured to be operated using signals from the controller 103. In some embodiments, the user support element 100 is a user profile structure configured to engage with a user perineal area and contain waste liquid generated during the cleaning process. In some instances, the sensor 101 may be configured to measure a user cleanliness state by using a user body surface measurement or a waste product measurement. A waste product measurement may include in some examples a measurement of a waste liquid, a cleaning tool or a cleaning wipe. In other embodiments, the sensor 101 may be configured to measure a user status, a cleaning element status, or an operator status. Examples of such statuses include a position of a user, a position of a cleaning element, and a position of an operator. In some embodiments, cleaning element 102 may include a nozzle configured to emit a liquid or a gas. In other embodiments, cleaning element 102 may include an electro-mechanical manipulator arm with an attached cleaning tool configured to contact and remove waste material from a user skin. In some embodiments, the manipulator arm may be capable of controller-directed operation independent of operator input while in others, it may be configured to be cooperatively commanded by an operator and the controller. In some embodiments, controller 103 may be configured to monitor and display a status of user cleanliness or operator cleaning function progress as measured by sensor 101. In such embodiments, controller 103 may not command kinetic or mechanical operation of user support element 100 or cleaning element 102.

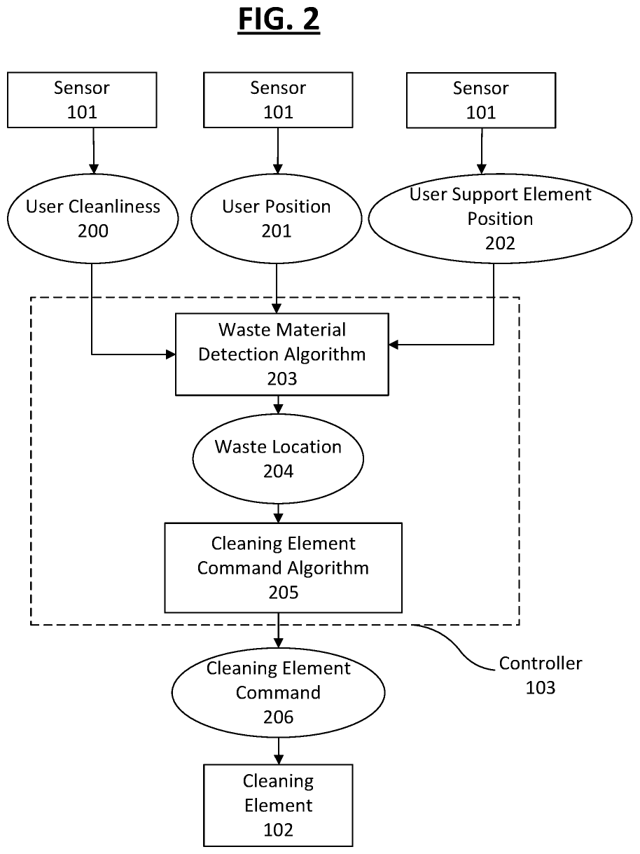

FIG. 2 depicts an example embodiment of perineal cleaning algorithmic inputs and outputs. Various data inputs and control algorithms may be utilized by a controller or command processing unit in combination with physical element instrumentalities. Sensors 101 generate data including user cleanliness data 200, user position data 201, and user support element position data 202. Said data is sent to a controller 103 and processed by a waste material detection algorithm 203. In some examples, user cleanliness data 200 may include the presence or location of waste material on a user body. User position data 201 may include the physical orientation and dimensions of a user body. In some examples, user support element position data 202 may include to the orientation or position of a user support element. The waste material detection algorithm 203 generates waste location data 204 including spatial coordinates of waste material. In some examples, waste material detection algorithm 203 may also generate a classification identity of detected waste material such as for example fecal matter or urine. The waste location data 204 is then delivered to a cleaning element command algorithm 205 where a cleaning element 102 is sent command data 206 generated by the command algorithm 205. Cleaning element command data 206 may include to a nozzle position, a liquid spray activation, or a cleaning tool course of travel in some instances.

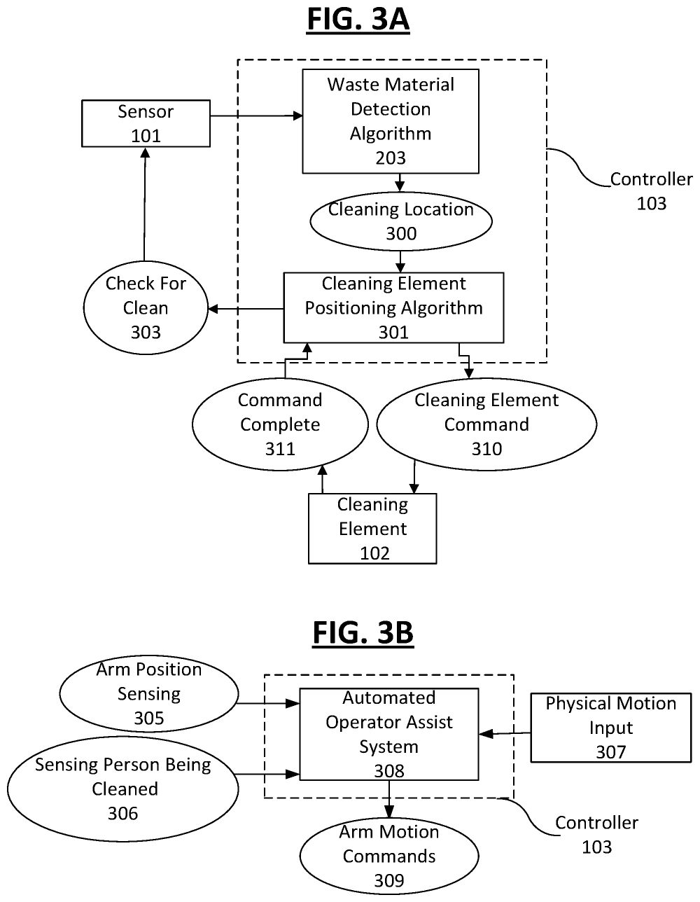

FIG. 3A is a diagram depicting a second example embodiment of perineal cleaning algorithmic inputs and outputs utilizing one or more sensors 101 to send a signal to a waste material detection algorithm 203 in a controller 103. Sensor 101 examples include visual, electro-optical, laser, infra-red, thermal, acoustic, or any other sensor capable of detecting and producing waste material location. In some embodiments, sensor 101 may detect and locate waste material directly on a user body. In other embodiments, sensor 101 may detect waste material originating from a user body indirectly, such as for example by monitoring waste material content in a generated waste stream. The waste material detection algorithm 203 then generates a cleaning location 300. In some embodiments, cleaning location 300 may include a coordinate for a cleaning function to occur or target. A cleaning element position algorithm 301 takes cleaning location 300 and communicates a cleaning element command 310 with a cleaning element 102. After executing the command 310, the cleaning element 102 transmits a command complete communication 311 back to the cleaning element positioning algorithm 301. The algorithm 301 or controller 103 then sends a check for clean command 303 to sensor 101 to check for the presence or location of waste material. In some embodiments, the sensor 101 remains activated so as to feed a continuous data signal to the waste material detection algorithm 203.

In some embodiments, a predetermined cleaning operation is performed prior to sensing or waste material detection algorithm functioning. In such an embodiment, a pre-scripted cleaning command may be sent to a cleaning element 102 to remove an initial level of waste material from a user. After a pre-scripted cleaning operation is complete, sensor 101 may then feed cleanliness information to waste material detection algorithm 203. Other predetermined cleaning operation examples may also include removing a user diaper, positioning a user, or any other repeatable, predictable or scriptable cleaning associated operations. In some embodiments, a predetermined cleaning operation may occur in parallel with sensing or waste material detection algorithm functioning. In other embodiments, a predetermined cleaning operation may occur following a sensing or waste material detection algorithm functioning.

FIG. 3B is a diagram depicting a third example embodiment of perineal cleaning algorithmic inputs and outputs utilizing an operator-electro-mechanical arm assisted approach to perineal cleaning. Arm position sensing information 305, user or person being cleaned sensing information 306, and physical motion inputs 307 are combined by an automated operator assist system 308 located within or embodied by a controller 103 to output electro-mechanical arm motion commands 309. Arm position sensing information 305 may include an arm proximity location relative to a user, feedback information generated by a physical force exerted on the arm, rotary position information, or any other information related to the state or position of an electro-mechanical arm. Such arm position sensing information 305 may be generated by any electrical, electro-mechanical, electro-optical sensor, or encoder. Sensing of a user being cleaned 306 may include body dimensions or coordinates, skin integrity or health status, or verbal or non-verbal communications in some examples. Physical motion input 307 may include the physical motions of a care giver operator exerted on a control element or surface in physical or operable connection with the electro-mechanical arm in some instances. Utilizing sensing data and signal inputs 305, 306, 307, the automated operator assist system 308 generates arm motion commands 309 including the examples of two-dimensional movement, three-dimension movement or cleaning tool activation.

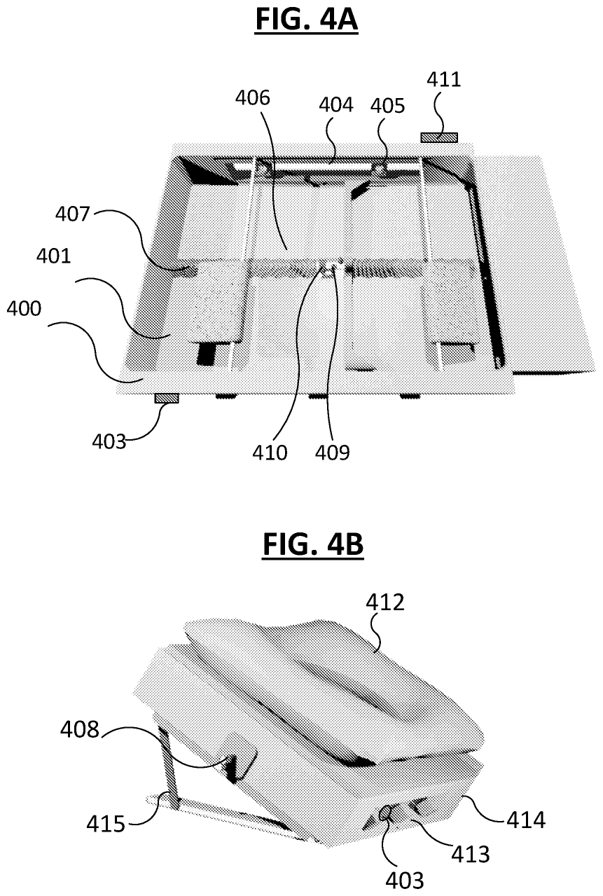

FIGS. 4A and 4B show an interior view and an angled profile view of an automated perineal cleaning apparatus embodiment, respectively. A containment structure 400 forms an interior compartment 401 having a drain 403 and a sensing window 404 that provides sensing access to the interior compartment 401. A sensor 405 is mounted behind sensing window 404 so as to protect sensor 405 from various contaminants including generated wastewater or user waste material. A cleaning element assembly 406 comprises a drive 407 mounted to the containment structure 400 and is configured to provide travel access to the interior compartment 401. The cleaning element assembly 406 further comprises an actuator 408 connected to the drive 407 and separated from the interior compartment 401 by the containment structure 400. The drive 407 may be covered by a protective sheath or covering so as to separate the drive from waste material, liquids, or other contaminants. The cleaning element assembly 406 also comprises a nozzle carriage 409 connected with the drive and including a nozzle 410 configured to emit a liquid or a gas. The embodiment also includes a controller 411 in connection with at least the actuator 408, the nozzle carriage 409, and the sensor 405. The controller 411 is configured to control elements of the cleaning element assembly 406 based on a signal from the sensor 405.

In some embodiments, the containment structure 400 forms a second sensing window 413 and includes a second sensor 414 mounted behind the second sensing window 413. In such an embodiment, sensors 405 and 414 work cooperatively to provide a broadened field of view of a user, as compared to an embodiment utilizing a single sensor. Other embodiments may include multiple other sensing windows and sensors in various orientations and arrangements relative to a user.

In an embodiment, the containment structure 400 includes a user profile structure 412 configured to engage with a user perineal area, support a user, and contain a waste generated or removed from user during a cleaning operation. The composition of the user profile structure 412 may vary depending on material selection considerations. Examples of materials comprising the user profile structure 412 include polymers, foams, silicones, ceramics, fiberglass composites, or stainless steel. In some embodiments, the physical structure of the user profile structure 412 is a static structure while in others, the structure is of tunable size or shape such as an inflatable structure. In some embodiments, the user profile structure 412 is formed according to the dimensions of a specific user to improve a cleaning seal and better support the user.

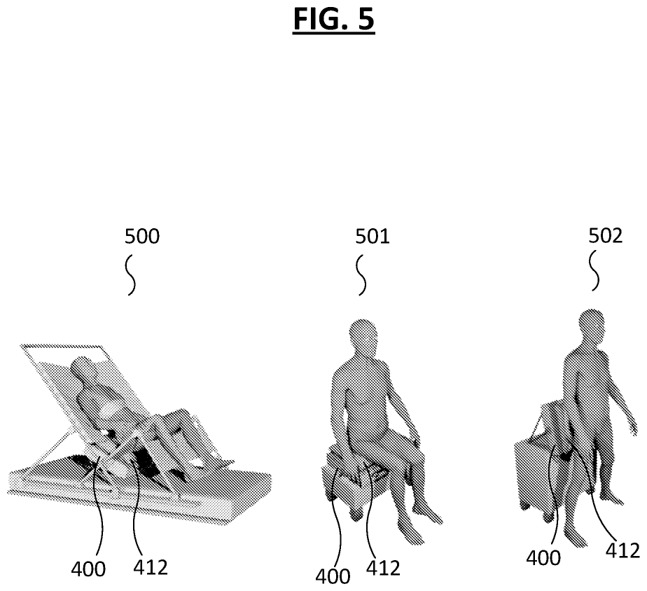

In an embodiment, the containment structure 400 includes an attached adjustable mount 415. In such an embodiment, the containment structure 400 as well as the user profile structure 412 attached to the containment structure 400 can be tunably positioned into a specific position or orientation relative to a user. Referring to FIG. 5 for an example, the containment structure 400 and user profile structure 412 may be tuned to engage with a user positioned in a reclined perineal cleaning position 500, a seated perineal cleaning position 501, or a standing perineal cleaning position 502.

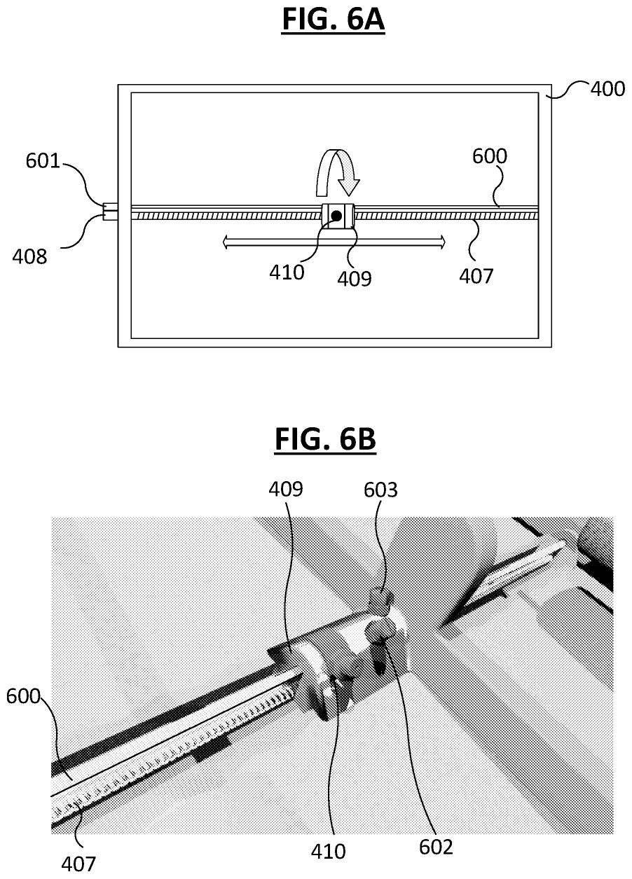

FIG. 6A illustrates an automated perineal cleaning apparatus embodiment utilizing a containment structure 400, a drive 407, an actuator 408 and a nozzle carriage 409 with a nozzle 410. Additionally, a rotation shaft 600 mounted to the containment structure 400, coextensive with the drive 407 and connected to the nozzle carriage 409 is included. The actuator 408 may jointly activate the drive 407 and the rotation shaft 600. An additional actuator 601 may also be included and configured to separately control the rotation shaft 600 independent of the drive 407. In such an example, actuator 408 actuates drive 407 and additional actuator 601 actuates rotation shaft 600. Actuators 408 and 601 may comprise an AC or DC motor, a brushless motor, a linear actuator, a servo, a pneumatic system, a hydraulic system, or any element capable of providing power. In such an embodiment, the controller 411 is configured to tune a position or orientation of the nozzle 410 using the drive 407 or rotation shaft 600 by operating the actuator 408 or 601. For an example, the nozzle carriage 409 traverses an axis of travel as guided by the drive 407 that is activated by the actuator 408 which as commanded by the controller 411. In another example, the rotational position of the nozzle 410 is tuned by the rotation shaft 601 activated by the actuator 601 and as commanded by the controller 411.

FIG. 6B depicts an example embodiment of perineal cleaning nozzles, drives, and rotation shafts. The nozzle carriage 409 may include multiple nozzles, 410, 602, and 603. In an embodiment, nozzles 602 and 603 are fixed to the nozzle carriage 409 such that the orientation of nozzles 602 and 603 is dictated by the position of the nozzle carriage 409 along drive 407. Nozzle 410 is rotatably mounted to the nozzle carriage 409 such that orientation of nozzle 410 may be adjusted by rotation shaft 600, independent of the position of nozzle carriage 409. Nozzles 410, 602, and 603 may have various aperture configurations. In one example, nozzle 410 is configured to emit a focused stream of liquid or gas and nozzles 602 and 603 are configured to emit a diffused or fan spray of liquid or gas.

In some examples, liquid or gas emission characteristics from nozzles 410, 602, or 603 may be tuned based on a signal from sensor 405 or command from controller 411. For example, the controller 411 may determine that a first user cleanliness status requires a first gas or liquid nozzle pressure, while a second user cleanliness status requires a second gas or liquid nozzle pressure. In another example, controller 411 may determine that a user skin integrity status requires a certain gas or liquid nozzle pressure. In another example, controller 411 may determine that a user body geometry or position relative to nozzles 410, 602, or 603 requires a certain gas or liquid nozzle pressure. In some examples, the temperature of the liquid or gas may be tuned based on sensor 405 or controller 411. Liquid or gas of higher or lower temperature may provide a user with a more comfortable experience, depending on preference. Additionally, liquid or gas of varying temperatures may aid in specific waste material removal function. For example, a fluid or gas of higher temperature may aid in dissolving solidified waste material from a user's body.

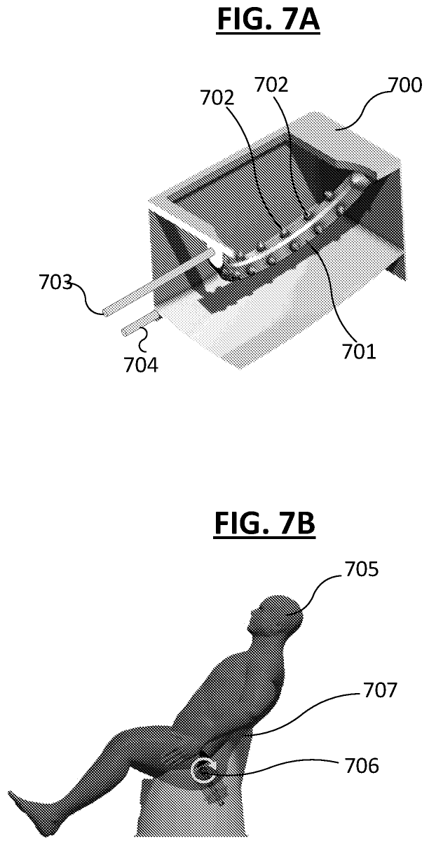

FIG. 7A illustrates an automated perineal cleaning apparatus embodiment including a containment structure 700 with an internally mounted rotating nozzle array 701 having a plurality of nozzles 702. The nozzle array 701 is configured to orient the nozzles 702 towards an operation window, such as towards a user perineal area. The nozzle array 701 forms an internal channel, connecting the nozzles 702 with a liquid or gas input line 703, such that liquid or gas can travel through the input line 703, into the nozzle array 701 and exit through the nozzles 702. The containment structure is connected with a liquid or gas exit line 704, such that liquid or gas collected in the containment structure may be removed through the exit line 704. The exit line 704 may rely on a gravitational, siphoning, or vacuum action to remove liquid or gas from the containment structure 700. As illustrated in the example of FIG. 7B, the mounted nozzle array 701 is configured to rotate on an axis 706, such that an emission of liquid or gas emitted from the nozzles 702 may be adjusted relative to a user 705. The containment structure 707 is formed so as to position and support the user 705 in a perineal cleaning position. In some embodiments, nozzle array 701 may rotate on axis 706 such that emitted liquid or gas may be used to clean elements of containment structure 700, such as during a post user cleaning sanitation process.

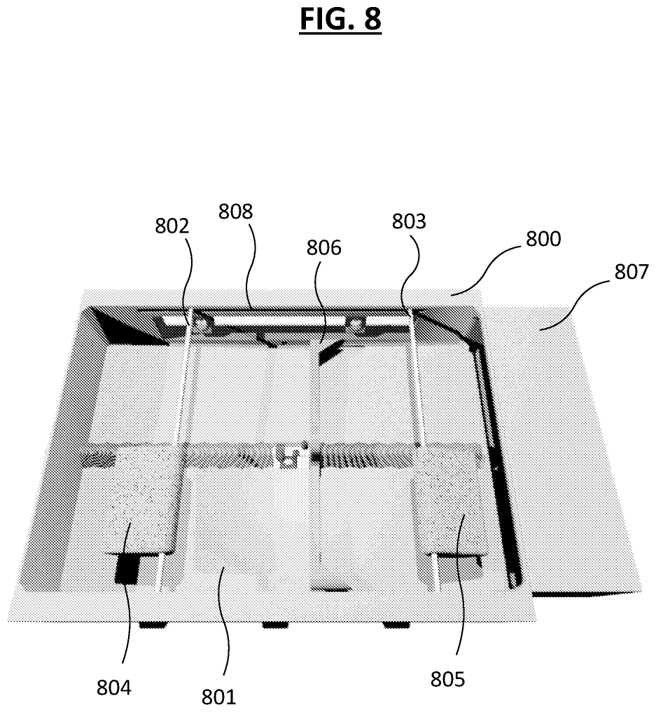

FIG. 8 depicts an internal view of an example automated perineal cleaning apparatus embodiment including a diaper manipulator, diaper pusher and diaper receptacle. An embodiment includes a containment structure 800 forming an interior compartment 801. A first rotating support 802 and a second rotating support 803 are mounted to the containment structure 800 and positioned adjacent to the interior compartment 801. A first diaper manipulator 804 is mounted to the first support 802 and second diaper manipulator 805 is mounted to the second support 803. The diaper manipulators 804, 805 are configured to remove a diaper article by rotational contact, and are shaped in a circular, oval, oblong, or elliptical geometry. In some embodiments, diaper manipulators 804, 805 may form any geometric structure capable of providing one or more contact points with a diaper or user, with at least one contact point positioned off centered from the rotating support 802 or 803 axis of rotation. Diaper manipulators 804, 805 may comprise various materials including polymers, foams, silicones, cork or other materials suitable for close, kinetic contact with a user body. Some embodiments include a diaper pusher 806 mounted within the interior compartment 801 and configured to traverse the compartment, providing the ability to move a diaper collected in the interior compartment 801. In an example, the diaper pusher 806 is attached to a linear track and drive system, such that activation of the drive moves the pusher 806 along the linear track. Diaper pusher 806 may be attached to an independent track and drive system, or attached to nozzle carriage 409 connected to drive 407 and actuated by actuator 408. A diaper receptacle 807 is in connection with the internal compartment 801 and is configured to receive and store diapers transferred by the diaper pusher 806. Rotating supports 802, 803 may be configured to traverse the interior compartment 801 so as to position diaper manipulators 804, 805 in variable locations along the interior compartment 801, including an active or nonactive location depending on whether a diaper removal function is required. In an example, the containment structure 800 forms a track 808 adjacent to and along a length of the interior compartment 801. Rotating supports 802, 803 are connected to the track 808 and configured to provide a diaper removal contact pressure by traversing the track 808 as driven by an electrically or manually powered actuator or drive. In such an example, the diaper manipulators 804, 805 are pressed against a diaper or user as a function of diaper manipulators' 804, 805 geometric shape rotating around a static axis of rotating supports 802, 803 or, by way of rotating supports 802, 803 providing contact pressure with a diaper or user by traversing toward one another along track 808.

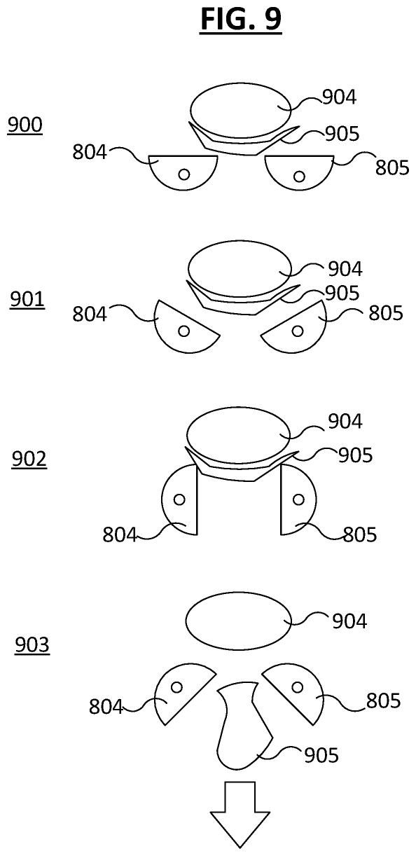

FIG. 9 depicts an example automated perineal cleaning apparatus embodiment including a diaper manipulator in various stages of operation. At 900, a user 904 wearing a diaper 905 is positioned adjacent to a first diaper manipulator 804 and a second diaper manipulator 805. At 901, diaper manipulators 804, 805 rotate toward diaper 905. At 902, diaper manipulators 804, 805 contact diaper 905 and exert rotational friction force against diaper 905. At 903, diaper manipulators 804, 805 continue rotation, removing diaper 905 from the user 904 and allowing the diaper 905 to fall away. The diaper 905 is beneficially shaped or molded as a result of the rotational action of the diaper manipulators 804, 805 so as to improve containment of waste material within the removed diaper 905.

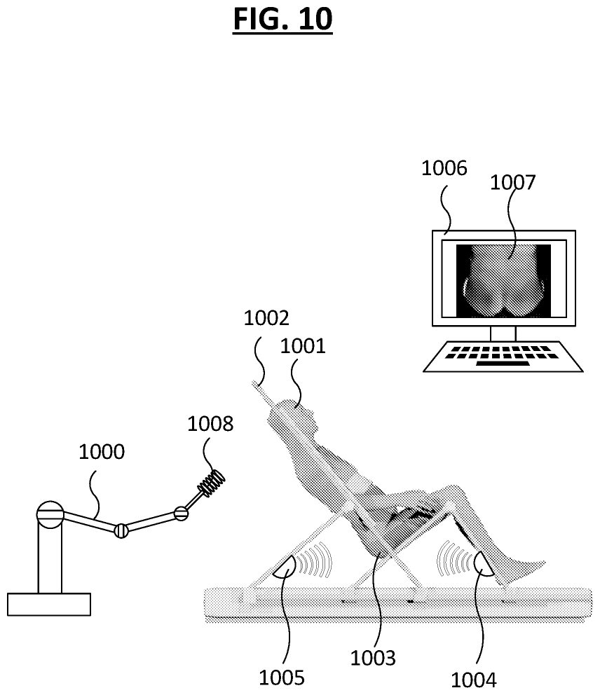

FIG. 10 illustrates an embodiment of a system of the present disclosure utilizing an electro-mechanical arm 1000 with an attached perineal area cleaning tool 1008. A user 1001 is positioned into a perineal cleaning position by a support element 1002 which allows for cleaning access to the user's perineal area 1003. Sensors 1004, 1005 are positioned on the user support element 1002 and are oriented to measure a cleanliness state of the user perineal area 1003. A wirelessly or wired connected controller 1006 collects signals from sensors 1004, 1005 and determines a cleanliness state of the user 1001. In some embodiments, the controller 1006 comprises a personal computer terminal having a human machine interface or display screen 1007. The controller 1006 is configured to display sensor readings collected from sensors 1004, 1005 or a status of electro-mechanical arm 1000 so that a care giver may monitor a status of a perineal cleaning function using display 1007. The controller 1006 then commands the elector-mechanical arm 1000 to perform cleaning operations based on the user 1001 cleanliness state. In various examples, the electro-mechanical arm 1000 comprises an articulated, six-axis, cartesian, rectangular coordinate, cylindrical coordinate, spherical coordinate, gantry, or any other form of electrically, hydraulically, or computer-based manipulation arm.

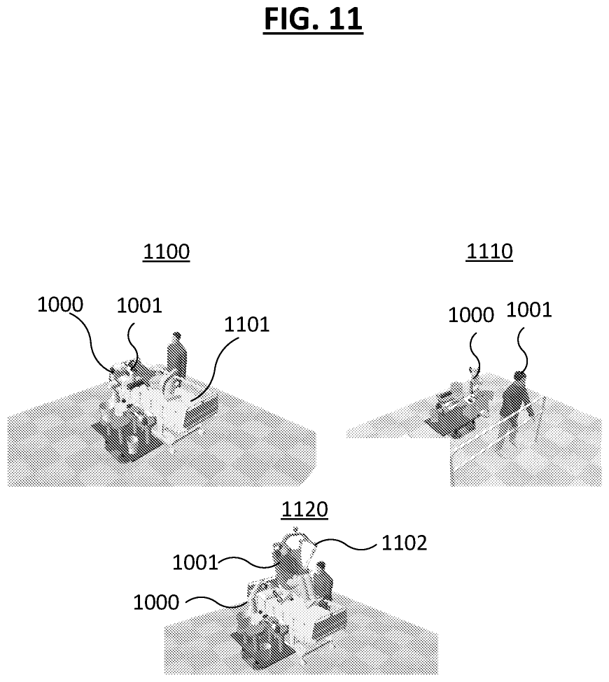

FIG. 11 depicts an example embodiment of the system of FIG. 10. The electro-mechanical arm 1000 and user 1001 may be positioned in various orientations with one another. At 1100, the user 1001 is positioned in a side-supported orientation on a bed 1101, such that the arm 1000 has cleaning access to the user's perineal area. At 1110, the user 1001 is positioned in a standing orientation such that the arm 1000 has cleaning access to the user's perineal area. At 1120, the user 1001 is positioned in a suspended orientation with the use of a sling element 1102 such that the arm 1000 has cleaning access to the user's perineal area.

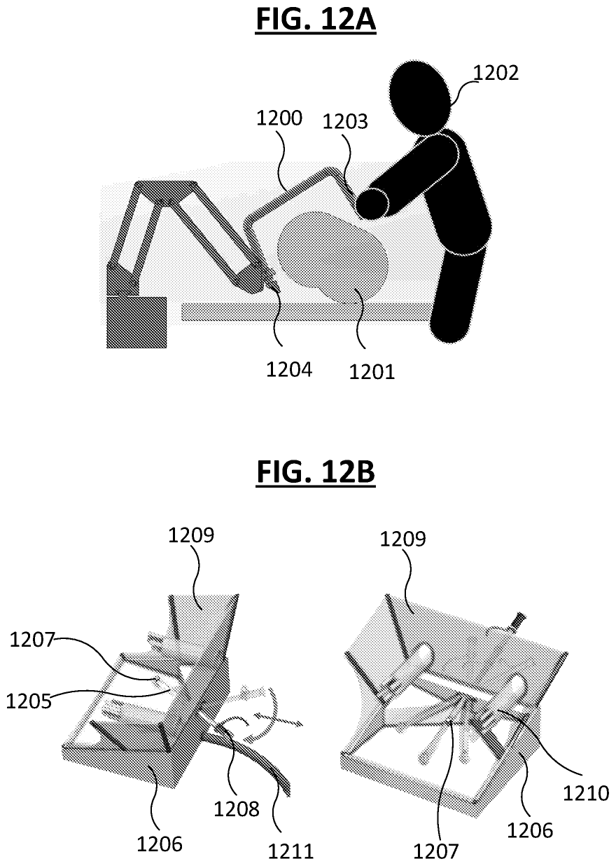

FIG. 12A depicts an example embodiment of an automated perineal cleaning apparatus including an electro-mechanical arm configured to work cooperatively with an operator to perform perineal cleaning. Electro-mechanical arm 1200 is positioned in a perineal cleaning orientation relative to a user 1201. An operator 1202 engages with the arm 1200 by a handle element 1203. The arm 1200 has an attached cleaning fixture 1204 such as a nozzle configured to emit liquid or gas or a mechanical cleaning tool in some examples. The cleaning fixture 1204 may also include a sensor capable of detecting or measuring waste material. A controller collects and generates information related to the user 1202 position, the arm 1200 position, the cleaning fixture 1204 position, the operator 1202 position, a user perineal cleanliness status, or a physical force exerted by the operator 1202 through the handle 1203. Such information may also include the dimensions of a safety space defined by the user's body spatial dimensions. If the controller determines that the cleaning fixture 1204 or arm 1200 penetrates the safety space for the user 1201, the controller may command the cleaning fixture 1204 or arm 1200 to deactivate or return to a determined safe location or state relative to the user 1201. Alternatively, the controller may determine that a force exerted on the arm 1200 through handle 1203 would result in the cleaning fixture 1204 or arm 1200 creating a hazardous condition or entering a user safety space. The controller may then command the arm 1200 to resist or counteract such operator 1202 force to prevent the hazardous condition or safety space breach from occurring.

FIG. 12B depicts an example embodiment of the automated perineal cleaning apparatus of FIG. 12A. An electro-mechanical arm 1205 is mounted to an angled support base 1206 and includes a cleaning tool 1207 and an operator handle 1208. A splash guard 1209 is attached to the angled support base 1206 so as to separate waste material from an operator. At least one integrated glove 1210 is connected to or formed by the splash guard 1209, such than an operator may insert his or her hand into the glove 1210 and manipulate a user through the glove 1210 without exposing him or herself to waste material. A drain line 1211 is connected to the support base 1206 and configured to remove generated waste liquids from the embodiment. The electro-mechanical arm 1205 may be connected to various electrical elements configured to command, tune or monitor functions of the arm 1205. In some embodiments, the arm 1205 includes a pressure sensor or force gauge configured to send a signal to a controller when contact between the cleaning tool 1207 and a user exceeds a threshold value. If the threshold value is breached, a controller may activate a warning element such as an alarm or haptic feedback system to notify an operator that a certain pressure or force threshold has been or is near being reached. In embodiments in which the arm 1205 is connected to a servo or other motor, the controller is configured to send commands to physically alter the position of the arm to reduce a measured pressure or force using the servo. In other embodiments, the connection point between the arm 1205 and the angled support base 1209 creates a reduced or specified range of motion for the arm 1205 and attached cleaning tool 1207.

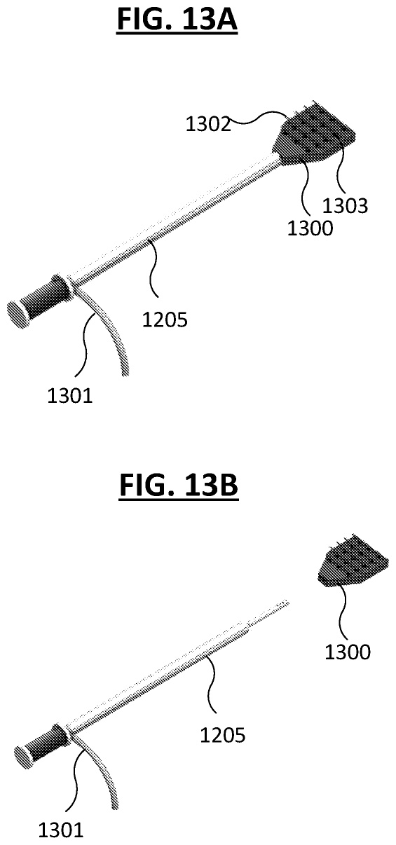

FIGS. 13A and 13B illustrate an example embodiment of an electro-mechanical arm with a removeable cleaning tool. The arm 1205 includes an internal structure, framework, or plumbing to transport a solid, fluid or liquid. An inlet line 1301 is connected to and configured to supply a fluid or liquid to the arm 1205. A removeable cleaning tool 1300 is attached to the arm 1205 and configured to emit the fluid or liquid. As increased contact surface area is beneficial to the removal of waste material from a user, the cleaning tool 1300 forms a ridged profile 1302, such that waste material is collected within the profile 1302. The cleaning tool 1300 includes or forms at least one liquid or gas port 1303 to emit a liquid or gas. The cleaning tool 1300 is comprised of example materials including a polymer, foam, silicone, cork, or other material suitable for close, kinetic contact with a user body.

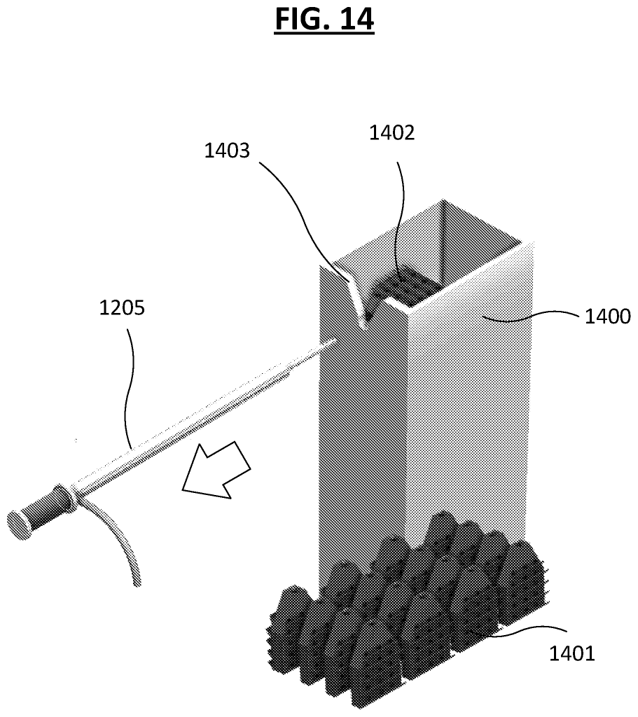

FIG. 14 depicts an example embodiment of the electro-mechanical arm of FIGS. 13A and 13B. The electro-mechanical arm 1205 includes or is positioned adjacent to a cleaning tool depository 1400. Factoring in cleaning tool material wear and biohazardous material handling procedures, it may be more hygienic and cost effective to limit the use of a single cleaning tool to one user. Accordingly, after a cleaning tool is used, it may be removed and conveniently stored in the depository 1400 to allow a new cleaning tool 1401 to be attached. The depository 1400 forms a detaching structure 1403 to engage with a used cleaning tool 1402, such that the cleaning tool 1402 is disconnected from the arm 1205 as the arm 1205 retracts away from the depository 1400. Alternatively, a used cleaning tool 1402 may be removed from the arm 1205 by a torsional, angular, or perpendicular force as the used cleaning tool 1402 remains engaged with the detaching structure 1403. The disconnected used cleaning tool 1402 may then fall inside the depository 1400 for disposal, after which a new cleaning tool 1401 can be selected and attached to the arm 1205 for further cleaning service.

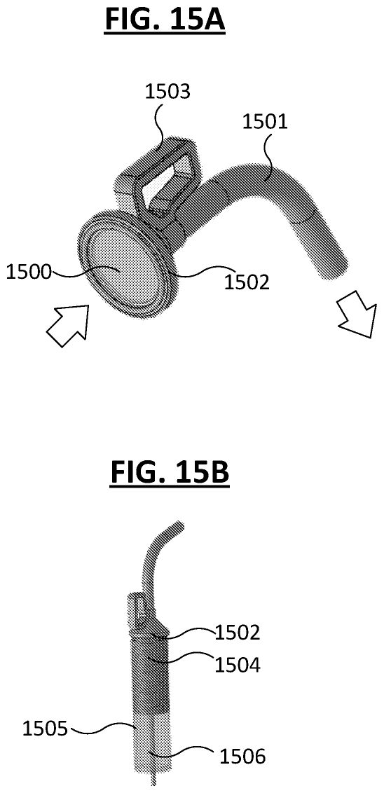

FIG. 15A and 15B depict an example embodiment of a cleaning tool including a waste collection suction pad. The waste collection suction pad 1500 is connected to a vacuum tube 1501 by a pad retention ring 1502. In some embodiments, the pad retention ring 1502 provides a base surface to which the collection pad 1500 acts against via suction force provided by the vacuum tube 1501. In other embodiments, the pad retention ring 1502 may physically engage with and lock to the pad retention ring 1502, thereby not requiring or relying on a suction force to retain the collection pad 1500 to the retention ring 1502. The vacuum tube 1501 includes an ergonomic handle 1503 to assist with operator manipulation of the collection pad 1500 during cleaning. The vacuum tube 1501 may be directly connected to a vacuum line or an electro-mechanical arm 1205. In embodiments where the vacuum tube 1501 is connected to an electro-mechanical arm 1205, the arm 1205 is connected to a vacuum line and configured to pull a vacuum via an internal structure or plumbing. As a suction force is applied through the waste collection pad 1500 via a vacuum or low-pressure area, waste material is pulled against the waste collection pad 1500, and liquid is separated from the waste material and pulled through the waste collection pad 1500 for downstream collection or disposal. Solid waste material too large to pass through the waste collection pad 1500 remains adhered to the pad via suction force and can be disposed of by removing the waste collection pad 1500 from the vacuum tube 1501 or electro-mechanical arm 1205. The waste collection pad 1500 is comprised of a woven, porous, semi-porous, permeable, or semi-permeable material or any material capable of selectively allowing waste liquid passing through the waste collection pad 1500.

Illustrated in FIG. 15B, new or reusable collection pads 1504 of some embodiments may be staged in a pad dispenser structure 1505. A suspension rod 1506 is mounted to and positioned within the dispenser structure 1505 and provides a force to ensure at least one new or reusable collection pad 1504 is seated in an exit stage of the dispenser structure 1505. In some examples, the suspension rod 1506 is electrically actuated or spring driven against the staged collection pads 1504. In other examples, suspension rod 1506 may be manually manipulated to position a new or reusable collection pad 1504. When a replacement collection pad 1504 is needed, the pad retention ring 1502 engages with and retains a new or reusable pad 1504 seated in the dispenser exit stage for cleaning service.

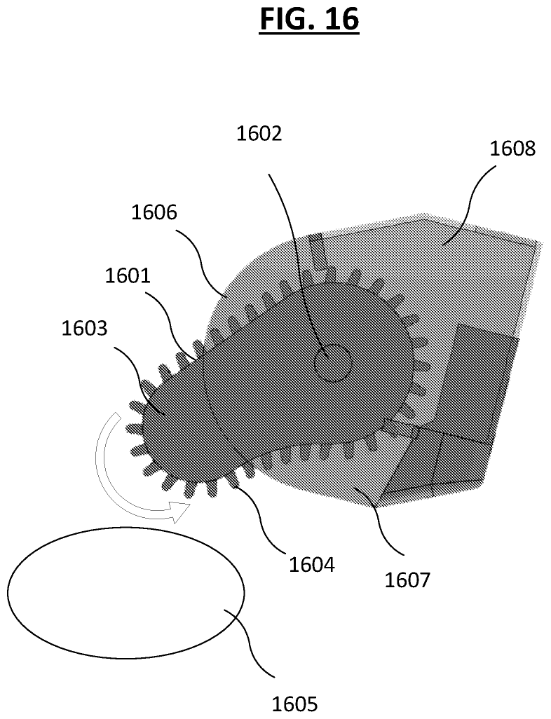

FIG. 16 depicts an example embodiment of a cleaning tool including a rotary wiper. The rotary wiper comprises a wiper belt 1601 mounted to a rotary axle 1602 such that rotation of the axle 1602 translates to linear motion of the wiper belt 1601 along a travel path defined by a track structure 1603 or mounting axle adjacent to the rotary axle. The wiper belt 1601 forms a plurality of wiping fingers 1604 shaped to remove waste material from a user 1605. As the wiper fingers 1604 are likely to contact a user skin, they may be comprised of various example materials including a polymer, rubber, silicone, foam, other material suitable for close, kinetic contact with a user body. The rotary axle 1602 is positioned within a containment housing 1606 forming a solid waste collection compartment 1607 and a sanitizing compartment 1608. As the rotary axle 1602 turns during cleaning operation, the wiper fingers 1604 remove waste material from a user 1605 and then enter the containment housing 1606. Waste material is removed from the wiper fingers 1604 or wiper belt 1601 and collected in the collection compartment 1607. Collection compartment 1607 may be connected to a drain or vacuum line to transfer collected waste material to a secondary collection location. In one example, waste material is removed from the wiper belt 1601 using a physical scraper mechanism or a liquid or gas flushing mechanism. The wiper belt 1601 next enters the sanitizing compartment 1607 where the wiper belt 1601 and fingers 1604 undergo a sanitizing process. Examples of a sanitizing process include applying a disinfecting chemical or solution or providing exposure to an ultraviolet light source. The sanitized wiper belt 1601 proceeds to exit the sanitizing compartment 1608 of the containment housing 1606, after which the wiper belt 1601 or fingers 1604 contact the user 1605 and remove additional waste material.

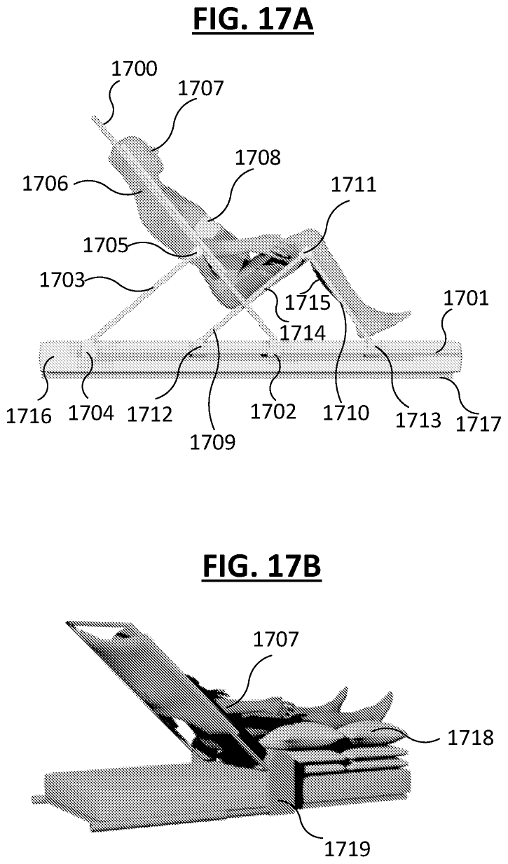

FIGS. 17A and 17B depict example embodiments of a user support element 100. A back support structure 1700 is connected to a linear motion track 1701 by a first sliding attachment 1702. A back support link 1703 is connected to the linear track 1701 by a fixed attachment 1704 and is further connected to the back support structure 1700 by a first joint 1705. The back support structure 1700 includes a user padding element 1706 capable of supporting a user 1707. A body strap 1708 is connected to the back support structure 1700 and is capable of securing a user 1707 during positioning or perineal cleaning. A foldable leg support structure is formed by a first 1709 and second 1710 leg support link attached to one another by a second joint 1711. The first leg support link 1709 is connected to the track 1701 by a second sliding attachment 1712 and the second leg support link 1710 is connected to the track 1701 by a third sliding attachment 1713. The first 1709 and second 1710 leg support link includes first 1714 and second 1715 leg padding elements capable of supporting a user 1707 thighs or legs respectively. A user perineal area is cooperatively positioned by the padding elements 1706, 1714 and 1715 to create sufficient clearance space to allow the perineal area to be accessed by a cleaning element 102 such as nozzle, a brush, or a wipe. An orientation of the back support structure 1700 and the foldable leg support structure can be adjusted or tuned by adjusting or tuning a linear position of the first 1702, second 1712, and third 1713 sliding attachments along the track. In some embodiments, the linear track 1701 or the sliding attachments 1702, 1712, 1713 include docking structures to lock the sliding attachments 1702, 1712, 1713 at a certain location along the track 1701. In some embodiments, the sliding attachments 1702, 1712, 1713 include a spring-mounted engagement member and the linear motion track 1701 includes at least one docking aperture in which an engagement member may enter, thereby preventing respective sliding attachments 1702, 1712, 1713 from moving along the track 1701. In some embodiments, sliding attachment movement and engagement member engagement are performed manually. In other embodiments, the sliding attachments 1702, 1712, 1713 are moved with the use of a connected electrical motor or servo commanded by a controller. As individuals requiring autonomous or assisted perineal care are often bed bound or more comfortably supported in or on a bed, the linear motion track 1701 may be mounted to a bed 1716. An under-bed support structure 1717 may also be positioned or mounted beneath the bed 1716 to provide structural support to the embodiment.

Illustrated in FIG. 17B, some user support element 100 embodiments utilize one or more inflatable structures 1718 to support or position a user 1707 into a perineal cleaning position. Embodiments may utilize inflatable structures 1718 exclusively, with inflatable structures 1718 positioned to support a user's 1707 upper body and lower body individually, while allowing for cleaning access to the user's 1707 perineal area. Other embodiments may utilize inflatable structures 1718 in combination with a back support structure 1700 or leg support links 1709, 1710. In some embodiments, the position or orientation of back support structure 1700 or leg support links 1709, 1710 is adjusted manually or with an electric motor or servo element 1719.

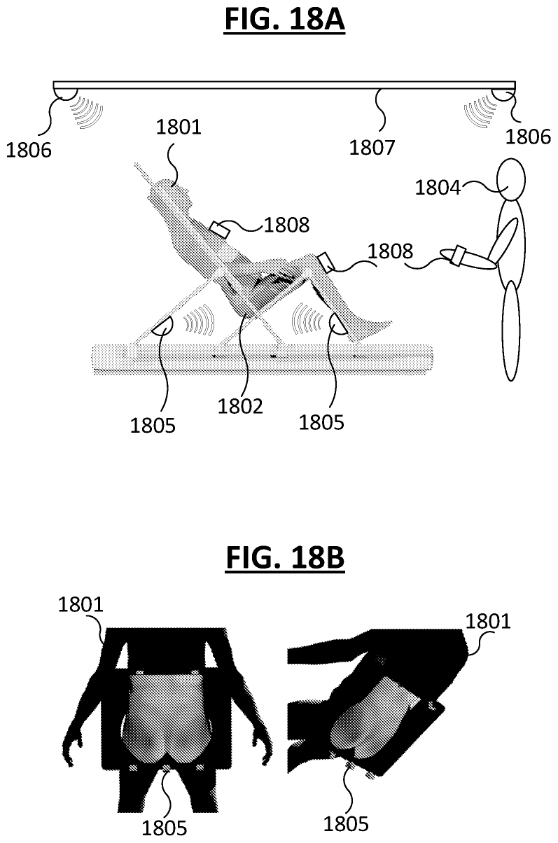

FIGS. 18A and 18B depict example embodiments of cleaning sensor arrangements. Embodiments may utilize sensors of various configurations relative to a user 1801, a user perineal area 1802, or an operator 1804. In an embodiment, sensors 1805 are attached to user support element 1803 and oriented toward a user perineal area 1802. Sensors 1806 are attached to an infrastructure element 1807. Sensors 1808 are attached directly to a user 1802 or operator 1804, for example by a wearable article. Depending on the type of sensor, location, and orientation of an embodiment, various types of information or data may be collected. In one embodiment, sensors 1806 detect the presence and body position of a user 1801 and an operator 1804. In another embodiment, sensors 1806 communicate with sensors 1808 to determine a position or orientation of a user 1801 or operator 1804. In another embodiment, sensors 1805 detect and measure a waste material on a user perineal area 1802. In another embodiment, sensors 1805, 1806 and 1808 positions or orientations are tunable, powered or manually, so as to provide varying field of views of user 1801, user perineal area 1802, or operator 1804. As illustrated in FIG. 18B, sensors 1805 may work cooperatively to form a single or multiple combined field of view. In some embodiments, sensors 1805, 1806 may detect and measure a skin condition of a user 1801, including for example the presence of rashes, sores, lesions, or cuts. By positioning sensors 1805 circumferentially around a user perineal area, the presence of waste material or lack thereof can be more accurately determined. In addition to utilizing sensors of varying reference angles, multiple types of sensors may also be utilized in combination. In an example, an ultrasonic sensor may be used in combination with an infrared sensor to form a single image or cleanliness profile of a user 1801. In another example, a visible camera sensor may be used in combination with a thermal camera sensor to form a single image or cleanliness profile of a user 1801. In another example, sensors tuned to varying image or data collection resolutions may form a single image or cleanliness profile of a user 1801.

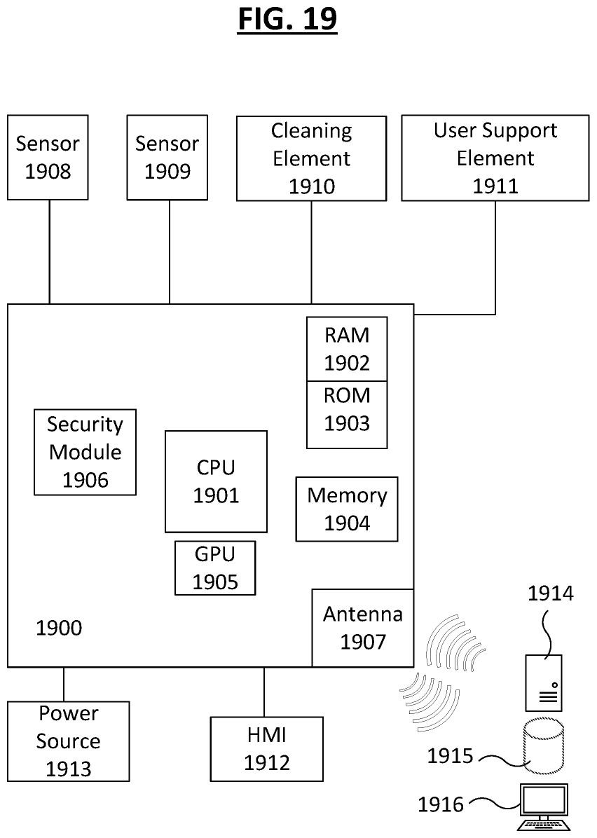

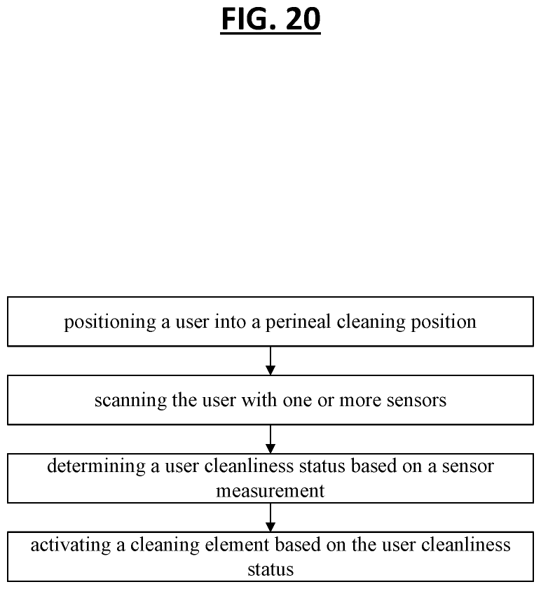

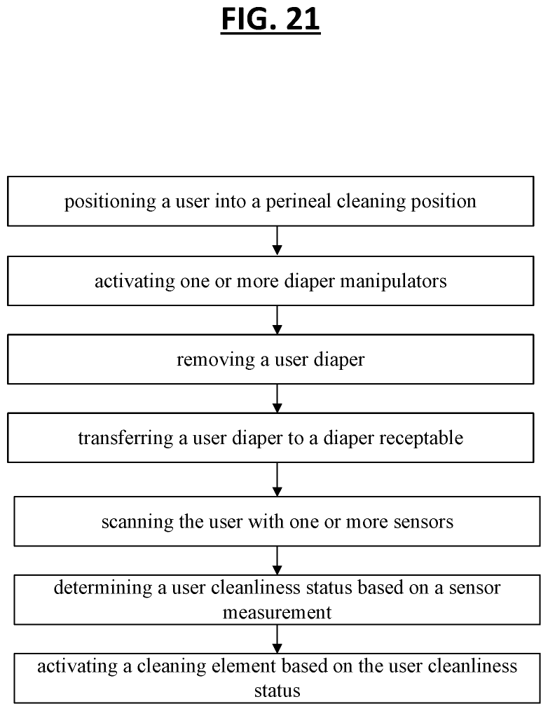

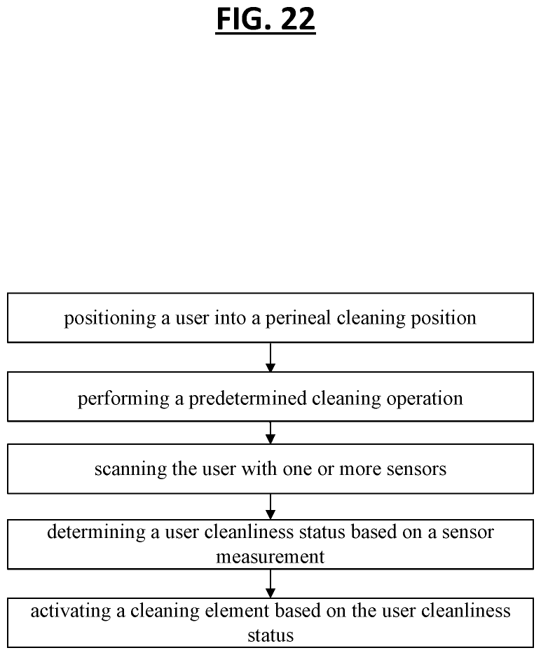

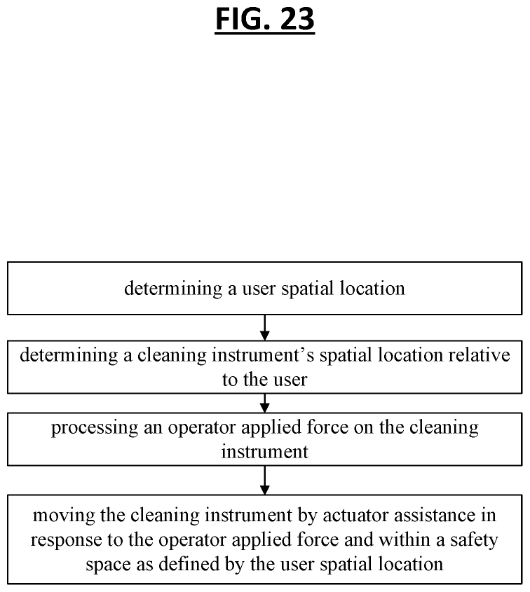

FIG. 19 depicts an example embodiment of a controller. A controller 1900 includes a CPU 1901, a RAM 1902, a ROM 1903, a memory storage 1904, a GPU 1905, a security module 1906, and an antenna 1907. The controller is wirelessly or physically connected to a first sensor 1908, a second sensor 1909, a cleaning element 1910, a user support element 1911, a human-machine interface (HMI) 1912, and a power source 1913. Using antenna 1907, the controller 1900 may communicate with a remote server 1914, database 1915, or command terminal 1916. In some embodiments, the controller 1900 may not rely on any external or remote commands or data, and may operate as an security-isolated system.

As user perineal cleaning data is inherently sensitive information, controller 1900 may utilize a security module 1906 to encrypt user data collected by connected elements such as sensors 1908 and 1909 in an embodiment. The controller 1900 may store encrypted user information in memory 1904, which may also include security portioned or siloed storage data spaces. The controller 1900 may also transmit encrypted user information to a remote database 1914, server 1915, or command terminal 1916. In some embodiments, encrypted user data remains solely within a secure environment of the controller 1900 for storage or algorithmic use, while non-user related data such as for example sensor 1908, cleaning element 1910, or user support element 1911 diagnostic data may be freely communicated to external locations.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

1. A system of automated perineal care comprising:
a user support element;
a sensor positioned on the user support element and configured to measure a cleanliness state of a user;
a cleaning element configured to clean a perineal area of the user; and
a controller configured to command operation of the cleaning element based on a signal from the sensor.

2. The system of claim 1, wherein the sensor is configured to detect and produce location data of waste matter on a body of a user, and wherein the controller is configured to command a position of the cleaning element based on the waste matter location data.

3. The system of claim 1, wherein the sensor is configured to have a tunable position, orientation or field of view.

4. The system of claim 1, wherein the cleaning element is i) a nozzle configured to emit a liquid or gas or ii) an electro-mechanical manipulator.

5. The system of claim 4, further comprising a cleaning tool attached to the manipulator and configured to contact a user and remove waste material from the user's skin.

6. The system of claim 5, wherein the cleaning tool is configured to be removable from the manipulator.

7. The system of claim 4, wherein the manipulator is configured to be cooperatively commanded by an operator and the controller.

8. The system of claim 1, wherein the user support element is a perineal cleaning positioning structure configured to provide cleaning access to a user's perineal area.

9. The system of claim 8, wherein the perineal cleaning positioning structure comprises a support frame or an inflatable structure.

10. A system of claim 1, wherein the user support element is configured to move based on signals from the controller.

11. The system of claim 1, wherein the sensor is configured to measure a user cleanliness state by using a user body surface measurement or a waste product measurement, wherein the waste product measurement is collected from a waste liquid, a cleaning tool, or a cleaning wipe.

12. The system of claim 1, further comprising a sensor configured to measure a user status, a cleaning element status, or an operator status.

13. An apparatus for automated perineal care comprising:
a containment structure forming an interior compartment having a drain;
a sensor mounted to the containment structure;
a cleaning element assembly comprising:
a drive mounted to the containment structure and configured to provide travel access to the interior compartment;
an actuator connected to the drive and separated from the interior compartment by the containment structure; and
a nozzle carriage connected with the drive and including a nozzle configured to emit a liquid or gas; and
a controller configured to the control the cleaning element assembly based on a signal from the sensor.

14. The apparatus of claim 13, wherein the containment structure forms a sensing window providing sensing access to the interior compartment, and wherein the sensor is mounted behind the sensing window.

15. The apparatus of claim 14, further comprising:
a second sensing window formed by the containment structure and providing sensing access to the interior compartment; and
a second sensor mounted behind the second sensing window, wherein first and second sensors behind the first and second sensing windows cooperatively form a field of view of a user.

16. The apparatus of claim 13, further comprising a rotation shaft mounted to the containment structure and coextensive with the drive and connected to the nozzle carriage, wherein the controller is configured to tune at least one of a position or orientation of the nozzle using the drive and rotation shaft.

17. The apparatus of claim 13, further comprising a second nozzle included on the nozzle carriage and configured to emit a focused stream of liquid or gas, wherein the at least one nozzle is configured to emit a fan spray of liquid or gas.

18. The apparatus of claim 13, further comprising a user profile structure attached to the containment structure and configured to engage with a user perineal area and contain a generated waste, the apparatus further comprising an adjustable mount attached to the containment structure and configured to tune an orientation of the containment structure.

19. An apparatus for perineal care comprising:
a containment structure forming an interior compartment;
a first and second rotating support mounted to the containment structure and positioned adjacent to the interior compartment; and
a first diaper manipulator mounted to the first support and a second diaper manipulator mounted to the second support, the diaper manipulators configured to remove a diaper article by rotational contact.

20. The apparatus of claim 19, wherein the containment structure forms a track adjacent to the interior compartment, and the first and second rotating supports are connected to the track and configured to provide a diaper removal contact pressure by traversing the track.

21. The apparatus of claim 19, further comprising a diaper receptacle in connection with the interior compartment and configured to receive a removed diaper, and further comprising a diaper pusher mounted within the interior compartment and configured to traverse the interior compartment and deposit a removed diaper within the diaper receptacle.

22. The apparatus of claim 19, wherein the first and second diaper manipulators each form a geometric shape configured to provide a contact point with a diaper or user, and wherein the contact point is positioned off-center of an axis of rotation of the first or second rotating supports.

23. An apparatus for perineal care positioning comprising:
a linear motion track;
a back support structure connected to the track by a first sliding attachment;
a back support link connected to the back support structure by a first joint and connected to the track by a fixed attachment; and
a foldable leg support structure formed by a first and second leg support link attached to one another by a second joint, the first leg support link connected to the track by a second sliding attachment and the second leg support link connected to the track by a third sliding attachment,
wherein an orientation of the back support structure and the foldable leg support structure is configured to be adjusted based on an adjustment to a linear position of the first, second, and third slidable attachments along the track.

24. The apparatus of claim 23, wherein the linear motion track is mounted to a bed.

25. A method of automated perineal care comprising the steps:
positioning a user into a perineal cleaning position;
scanning the user with one or more sensors;
determining a user cleanliness status based on a sensor measurement; and
activating a cleaning element based on the user cleanliness status.

26. The method of claim 25, wherein determining a user cleanliness status comprises identifying one or a combination of the presence or location of waste matter on a user body and wherein activating a cleaning element comprises tuning a cleaning element to a position or orientation corresponding to the identified waste matter.

27. The method of claim 25, further comprising the steps of:
activating one or more diaper manipulators;
removing a user diaper; and
transferring a removed diaper to a diaper receptable.

28. The method of claim 25, further comprising the step of performing a predetermined cleaning operation.

29. The method of claim 25, wherein determining a user cleanliness status comprises one or a combination of digital image processing or waste water analysis.

30. A method of automated perineal care comprising the steps:
determining a user spatial location;
determining a cleaning instrument's spatial location relative to the user;
processing an operator applied force on the cleaning instrument; and
moving the cleaning instrument by actuator assistance in response to the operator applied force and within a safety space as defined by the user's spatial location.
